(19) United States
(12) Patent Application Publication
(10) Pub. No.: US 2024/0115113 A1
(43) Pub. Date: Apr. 11, 2024

(54) FLUID MANAGEMENT WITH ABNORMALITY DETECTION

(71) Applicants: William E. Marvin, III, Attleboro, MA (US); Jake Terravecchia, Brighton, MA (US)

(72) Inventors: William E. Marvin, III, Attleboro, MA (US); Jake Terravecchia, Brighton, MA (US)

(21) Appl. No.: 18/483,610

(22) Filed: Oct. 10, 2023

Related U.S. Application Data
(60) Provisional application No. 63/379,071, filed on Oct. 11, 2022.

(51) Int. Cl. A61B 1/00; A61B 1/015

(52) U.S. Cl. CPC A61B 1/00057; A61B 1/015; A61B 2217/005; A61B 2217/007

(57) ABSTRACT

A system for detecting abnormalities in medical devices during medical procedures within a patient can include an inflow tubing defining an inflow lumen configured to provide a fluid from a fluid source to a site of the medical procedure. An inflow sensor can be in communication with the inflow lumen to sense an inflow parameter of the fluid within the inflow lumen. An outflow tubing can define an outflow lumen configured to extract debris from the site of the medical procedure. An outflow sensor can be in communication with the outflow lumen to sense an outflow parameter of the debris within the outflow lumen. A memory including instructions; and processing circuitry that, when in operation, can be configured by the instructions to identify an abnormality in either the inflow tubing or the outflow tubing based on comparing the inflow signal or the outflow signal, respectively, to a pre-determined threshold.

### CLAIM OF PRIORITY

This patent application claims the benefit of priority, under 35 U.S.C. Section 119(e), to Jake Terravecchia et al., U.S. Patent Application Ser. No. 63/379,071, entitled “PUMP TUBESET DEVELOPMENT,” filed on Oct. 11, 2022 (Attorney Docket No. 5409.820PRV), which is hereby incorporated by reference herein in its entirety.

### TECHNICAL FIELD

Examples described herein generally relate to medical pumping systems. More specifically, examples described herein generally relate to pumping systems including fluid management with abnormality detection.

### BACKGROUND

Endoscopes are typically used to provide access to an internal location of a patient so that a doctor is provided with visual access. Some endoscopes are used in minimally invasive surgery to remove unwanted tissue or foreign objects from the body of the patient. For example, an endoscopic tissue removal device is an instrument used by a clinician to remotely access necrotic, cancerous, damaged, infected, or otherwise unwanted soft tissue, bone, or other anatomical structures at an anatomical site, excise said unwanted matters from the adjacent anatomy, and transport them away from the anatomical site.

Some endoscopes have suction channels (also known as aspiration channels) to transport resected tissue, calculi (e.g., stones or stone fragments in various stone-forming regions), and mass, among other unwanted matters. A flow of irrigation agent (e.g., saline solution) can be introduced to the anatomical site through an irrigation channel in the endoscope during the procedure. The irrigation fluid can facilitate removal of the tissue debris, stone fragments, and other unwanted matters through the suction channel. The irrigation fluid can also help maintain a clear visibility of the anatomical environment for the clinician performing the procedure. Additionally, the irrigation flow can have a cooling effect on the endoscopic tissue removal device to help dissipate the heat generated during ablation of tissue or calculi (e.g., kidney stones).

The irrigation tubing can become kinked or develop a leak during the medical procedure. Moreover, unwanted matters produced during an endoscopic procedure can accumulate and clog a working channel (e.g., a suction channel) of the scope. Monitoring channels for clogging, and timely and efficiently unclogging the obstructed channel can reduce procedure time and improve efficiency, safety, and success of endoscopic procedures.

### SUMMARY

In examples, a system for detecting abnormalities in a medical device during a medical procedure in a patient can include an inflow tubing defining an inflow lumen. The inflow lumen can be configured to provide a fluid from a fluid source to a site of the medical procedure. An inflow sensor can be in communication with the inflow lumen to sense an inflow parameter of the fluid within the inflow lumen. The inflow sensor can be configured to generate an inflow signal indicative of the inflow parameter of the fluid. An outflow tubing can define an outflow lumen. The outflow lumen can be configured to extract debris from the site of the medical procedure. An outflow sensor can be in communication with the outflow lumen to sense an outflow parameter of the debris within the outflow lumen. The outflow sensor can be configured to generate an outflow signal indicative of the outflow parameter of the debris. The system can also include a memory including instructions; and processing circuitry that, when in operation, is configured by the instructions to: receive at least one of the inflow signal or the outflow signal; and identify an abnormality in either the inflow tubing or the outflow tubing based on comparing the inflow signal or the outflow signal, respectively, to a pre-determined threshold.

In examples, a method for detecting abnormalities in a medical device during a medical procedure in a patient can include receiving, from an inflow sensor, an inflow signal indicative of one or more inflow parameters of a fluid within an inflow lumen. Receiving, from an outflow sensor, an outflow signal indicative of one or more outflow parameters of debris within an outflow lumen. Identifying an abnormality in the inflow lumen or the outflow lumen by comparing the inflow signal or the outflow signal to an inflow pre-determined threshold and an outflow pre-determined threshold, respectively. The method can also include generating an inflow error or an outflow error based on the abnormality in the inflow lumen or the outflow lumen, respectively.

### BRIEF DESCRIPTION OF THE DRAWINGS

Various examples are illustrated in the figures of the accompanying drawings. Such examples are demonstrative and not intended to be exhaustive or exclusive examples of the present subject matter.

FIG. 1 is a diagram illustrating an example of an endoscope including an example system for fluid management with abnormality detection.

FIG. 2 is a block diagram of an example of a system for fluid management with abnormality detection.

FIG. 3 is a schematic block diagram of an example of a portion of an example system for fluid management with abnormality detection.

FIGS. 4-9 are graphical representations of example reference lines including buffer zones for example fluid management systems with abnormality detection.

FIG. 10 is a schematic diagram of an example method for fluid management systems with abnormality detection.

FIG. 11 is a schematic diagram of an exemplary computer-based clinical decision support system (CDSS).

FIG. 12 is a block diagram illustrating an example of a machine upon which one or more examples can be implemented.

### DETAILED DESCRIPTION

An endoscope can include a tubular portion (elongated member) insertable into an interior of an organ or a cavity (or lumen) of the body to assist in diagnosis or treatment. One or more working channels (e.g., a suction channel or an irrigation channel) can be disposed inside and extend along a length of the tubular portion. To lower the risk of unintentionally engaging with unintended tissue, the insertable tubular portion can have a smaller diameter than the rest of the endoscope. Consequently, the working channels can also have small lumen diameters. Therefore, because of the small diameters of the working channels, tissue debris and foreign objects (e.g., calculi and fragments thereof) can accumulate and clog the working channel.

A fluid management system can be connected to the endoscope to provide an irrigation fluid (e.g., saline) and suction to the endoscope. The fluid management system can include inflow tubing and outflow tubing fluidically connected to the irrigation channel and the suction channel of the endoscope, respectively. Similar to the working channels, the inflow tubing and the outflow tubing can become clogged, kinked, or blocked.

In this document, “clog” refers to tissue debris, calculi (e.g., kidney stones or stone fragments), and other matter that can accumulate and block the lumen of a channel partially or completely, “clogging” refers to a state of partial or complete blockage of the channel lumen, “kink” refers to tubing bending, warping, or becoming deformed such as to interfere with fluid or debris flow therethrough, and “leak” refers to damage to the tubing such that the liquid (e.g., irrigation fluid or debris) within the tube is leaking. Clogging can occur in any working channel of an endoscope.

Clogging in a suction channel or the outflow tubing can significantly reduce the efficiency of removing tissue debris and stone fragments therethrough. Delayed or inefficient removal of unwanted matters from the anatomical site can inhibit or prevent further treatment (e.g., debridement or ablation of stones), contaminate the anatomical site, or expose the patient to an increased risk. On the other hand, clogging in an irrigation channel or inflow tubing can reduce the volume or the flow rate of irrigation fluid flowing therethrough and supplied to the anatomical environment. The slow irrigation flow can be less efficient in flushing out unwanted matters from the anatomical site and increase the likelihood of clogging in the suction channel. Reduced irrigation volume and flow rate can also affect its cooling effect on the surgical members and the anatomical environment and increase the chance of heat accumulation at the anatomical site. Moreover, clogging in any working channel can block the lens of the endoscope, impair the visibility of the object under inspection, and reduce the quality of images taken in the anatomical environment, thereby increasing procedure difficulty and time.

Various approaches have been attempted to prevent or resolve channel clogging in endoscopes. For example, breaking unwanted matters (e.g., tissue debris or stone fragments) into finer pieces can reduce the likelihood of being clogged in the channel. This, however, can consume more energy, take a longer procedure time, and potentially increase patient risk due to the added procedure complexity and time. Fine particles or stone dust can reduce the visibility of the surgical field. Conventionally, unclogging is usually performed externally, which requires a clinician to retract the scope from the body, flush the obstructed scope to unclog it, and insert it back into the anatomical site. This approach increases procedure time, adds inconvenience to the clinician, and can increase surgical risks for the patient. In situ unclogging of a working channel when the endoscope remains inserted and held in position generally requires high-pressure irrigation, which can impose excessive positive pressure on the internal organ.

The inventors of the present disclosure have recognized an unmet need of endoscopic systems capable of monitoring the irrigation and suction lines to determine anomalies (e.g., kinks or leaks in tubing, blockages or clogs, or the like) in such lines.

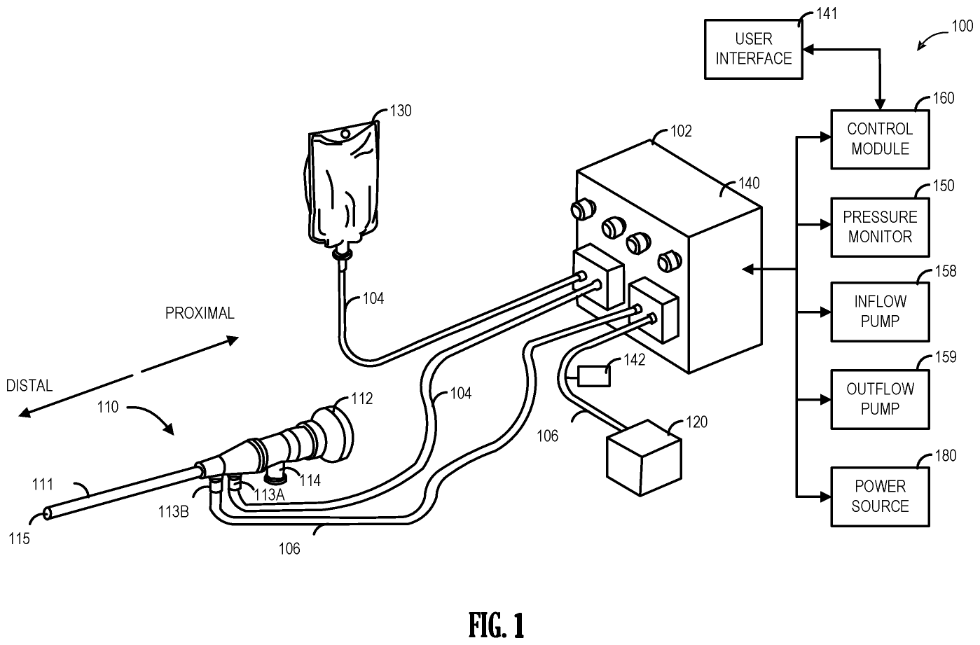

Disclosed herein systems and methods of fluid management with abnormality detection for use with endoscopes. The fluid management systems with abnormality detection for use with endoscopes or other medical systems will be discussed herein with reference to FIGS. 1-12.

FIG. 1 is a diagram illustrating an example of an endoscopic system 100 including an example fluid management system 102 with abnormality detection and an endoscope 110. The fluid management system 102 can include an inflow tubing set (inflow tubing 104), an outflow tubing set (outflow tubing 106), a suction source 120, an irrigation source 130, and a suction/irrigation control unit 140.

The endoscope 110, can extend into a sheath including a tube 111 extending from a distal end to a hub 112. The hub 112 can terminate at a proximal end. The endoscopic system 100 can include a light port 114 and a visual port 115. The light port 114 can function to provide light into the endoscope, and out of the tube 111 such that a feature of interest in the anatomical environment (e.g., resected tissue or calculi and matter) can be illuminated. The light port 114 can enhance visibility, for instance, when the feature of interest is located in low light conditions. The visual port 115 can function to provide a viewing window that allows a user to observe a feature of interest. The visual port 115 can be an optical window at the proximal end that provides visual access to a viewing lens at the distal end. In another example, the visual port 115 can provide a connection point to a camera to take images or video of the feature of interest and the anatomical environment. The images or video can be output and displayed on a monitor. In yet another example, the visual port 115 can include a camera. The camera can be integral to the visual port 115 and one or more lights can be positioned proximate to the camera such as to provide light in front of the cameras. In examples, the one or more lights can get their energy from a light source within the handle of the endoscope 110.

The endoscope 110 can include an irrigation/suction port 113 for receiving suction or irrigation fluid. The irrigation/suction port 113 can be located on an exterior of the hub 112, or other locations on the endoscopic system 100, such as a proximal end of the endoscopic system 100. The irrigation/suction port 113 can be a single connection point or can be separate connection points (113A and 113B) including one to connect to the inflow tubing 104 and the other to connect to the outflow tubing 106 to the working channel of the tube 111. The irrigation/suction port 113 can be open to a working channel inside the tube 111. The working channel can be sized, shaped, and configured to transport irrigation fluid or for suction. The same working channel can be used for irrigation and suction (also referred to as a unified irrigation/suction channel). In another example, an irrigation channel and a suction channel can be separately disposed within the tube 111.

The suction/irrigation control unit 140 can provide suction and irrigation to the endoscopic system 100 during an endoscopic procedure, while keeping the pressure of the anatomical environment under control, such as by maintaining the pressure substantially at a user-specified pressure level (e.g., the user-specified pressure with a tolerance such as ±5-10%). The suction/irrigation control unit 140 can include a pressure monitor 150, a control module 160, an inflow pump 158, an outflow pump 159, and a power source 180. The control module 160 can be in communication with a user interface 141 such as located on an exterior of the suction/irrigation control unit 140, for controlling the control module 160.

The suction source 120 can be connected to suction/irrigation control unit 140 via the outflow tubing 106. The suction/irrigation control unit 140 can include a control valve 142 configured to control the suction between the suction source 120 and the irrigation control unit 140 so that suction can be turned off during all or a portion of the application cycle of the irrigation fluid. The irrigation source 130 can be connected to the suction/irrigation control unit 140 via the inflow tubing 104. The inflow pump 158 and the control module 160 can each be included in or controlled by the suction/irrigation control unit 140 to pressurize the irrigation fluid before entering the endoscopic system 100 via the inflow tubing 104 and extract debris from the endoscopic system 100 via the outflow tubing 106. The inflow tubing 104 and the outflow tubing 106 can be connected at a common fitting, which can be coupled to a common line for supplying the fluid or suction to the endoscope 110 via a single irrigation/suction port 113.

The control module 160 can be configured to control the operation of the endoscope 110 in response to user commands from the user interface 141, detected parameters of either the inflow tubing 104 or the outflow tubing 106, detected characteristics of the inflow pump 158 or the outflow pump 159, or any other status of any of the components of the endoscopic system 100. The control module 160 can detect abnormalities in either the inflow tubing or the outflow tubing (e.g., a unified irrigation/suction channel, a separate irrigation channel, or a separate suction channel) based on an inflow parameter (e.g., a voltage supplied to either of the inflow pump 158 or the outflow pump 159, pressure within the inflow tubing 104, pressure of fluid supplied to the inflow tubing 104, or the like), or an outflow parameter (e.g., voltage supplied to the outflow pump 159, pressure within the outflow tubing 106, pressure of fluid supplied to the outflow tubing 106, or the like), respectively, and alert (e.g., with a warning, signal, alarm, or the like) the clinician, or send one or more control signals to any of the components of the endoscopic system 100 to remedy the detected abnormality in the endoscopic system 100. The control module 160 can automatically adjust one or more irrigation flow parameters or one or more suction flow parameters to keep the pressure of the anatomical environment (the “environmental pressure”) under control, such as to maintain the environmental pressure at substantially a user-specified pressure level.

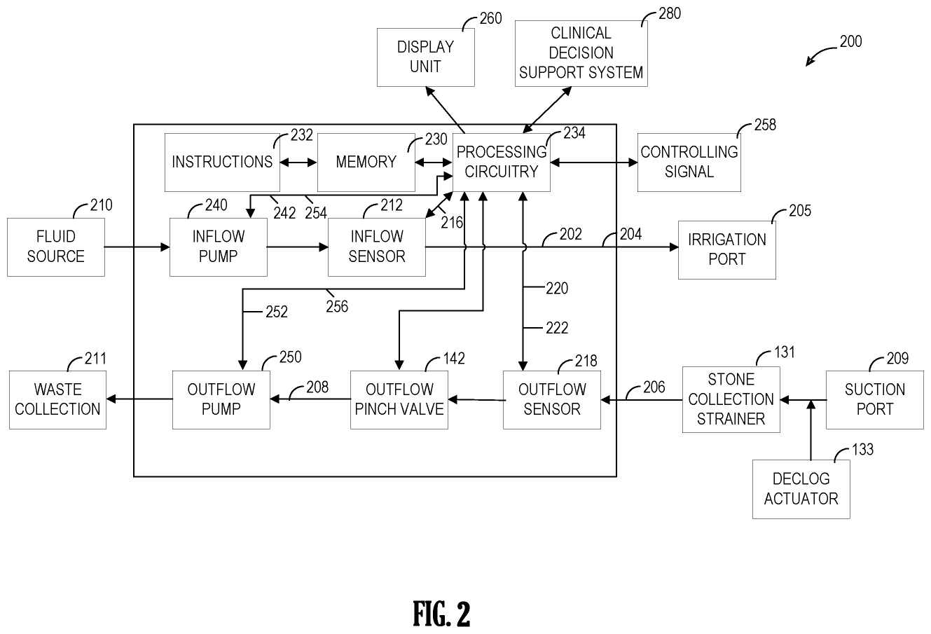

FIG. 2 is a block diagram of an example of a system 200 configured to manage fluid through an endoscopic system (e.g., the endoscopic system 100 (FIG. 1)) and detect abnormalities within the fluid management system (e.g., the fluid management system 102 (FIG. 1)). The system 200 can include an inflow tubing 202 and an outflow tubing 206.

The inflow tubing 202 can define an inflow lumen 204. The inflow lumen 204 can be configured to provide a fluid (e.g., irrigation liquid) from a fluid source 210 to a site of the medical procedure (e.g., a distal tip of the tube 111 (FIG. 1)) via the irrigation port 205 (e.g., the irrigation port 113A (FIG. 1)). The system 200 can also include an inflow sensor 212. The inflow sensor 212 can be a flow meter, optical sensor, thermometer or thermocouple, viscometer, or the like. The inflow sensor 212 can be in communication with (e.g., have at least a portion extending within the inflow lumen 204 or in fluidic communication with the inflow lumen 204, or the like) to sense or detect an inflow parameter 214 of the fluid within the inflow lumen 204. The inflow sensor 212 can be configured to generate an inflow signal 216, which can be indicative of the inflow parameter 214 of the fluid within the inflow lumen 204.

An inflow pump 240 (e.g., the inflow pump 158) can be in communication with the inflow tubing 202 to control a fluid flow rate of the fluid through the inflow tubing 202 or the inflow lumen 204. The inflow pump 240 can be controlled by adjusting an inflow pump voltage 242. In examples, the inflow parameter 214 can be either the inflow pump voltage 242, the fluid flow rate 254, any other parameter of the fluid within the inflow tubing 202 (e.g., temperature, viscosity, clarity, or the like), or the like.

The outflow tubing 206 can define an outflow lumen 208. The outflow tubing 206 can be configured to extract debris (e.g., excess liquid, ablated portions of tissue, fragments of stones or other organic matter, or the like) from the site of the medical procedure (e.g., the distal tip of the tube 111 (FIG. 1)) via the suction port 209 (e.g., the suction port 113B (FIG. 1) and into a waste collection 211. The system 200 can also include a stone collection strainer 131 and a declog actuator 133 to collect stones and remove larger chunks of debris from within the outflow lumen 208. In examples, the declog actuator 133 can be configured to help clear clogs or blockages within the outflow lumen 208.

An outflow sensor 218 can be a flow meter, optical sensor, thermometer or thermocouple, viscometer, or the like. The outflow sensor 218 can be in communication with (e.g., have at least a portion extending within, fluidic communication, or the like) the outflow lumen 208 to sense or detect an outflow parameter 220 of the fluid within the outflow lumen 208. The outflow sensor 218 can be configured to generate an outflow signal 222, which can be indicative of the outflow parameter 220 of the fluid within the outflow lumen 208.

An outflow pump 250 can be in communication with the outflow tubing 206 to control a debris flow rate 256 of the debris through the outflow lumen 208. The outflow pump 250 can be controlled by adjusting an outflow pump voltage 252. In examples, the outflow parameter 220 can be either the outflow pump voltage 252, the debris flow rate 256, any other parameter of the debris within the outflow tubing 206 (e.g., temperature, viscosity, clarity, or the like), or the like.

The system 200 can also include a memory 230 and processing circuitry 234. The memory 230 can include instructions 232. When in operation, the processing circuitry 234 is configured by the instructions 232 to receive at least one of the signals (e.g., the inflow signal 216 or the outflow signal 222) and identify an abnormality in either of the inflow tubing 202, the outflow tubing 206, or within the tube 111 (FIG. 1) or any other component of the endoscopic system 100 (FIG. 1) based on comparing the inflow signal 216 or the outflow signal 222, respectively, to pre-determined thresholds.

In examples, the processing circuitry 234 can generate a controlling signal 258. The controlling signal 258 can be transmitted to any component of the system 200 such as to change an operating parameter of that component to adjust either of the inflow parameter 214, the outflow parameter 220, fluid flow rate 254, or the debris flow rate 256 based on an abnormality detected in either of the inflow lumen 204 or the outflow lumen 208. For example, the controlling signal 258 can be configured to alter a voltage supplied to either of the inflow pump 240 or the outflow pump 250 to alter their pumping rates of the irrigation liquid and debris, respectively.

As will be discussed herein, the processing circuitry 234 can be connected to a display unit 260 and a clinical decision support system (CDSS 280). The display unit 260 can be configured to alert the clinician of any abnormalities detected by the system 200 and the CDSS 280 can be configured to help the clinician in their procedures and diagnoses of the patient during the medical procedure.

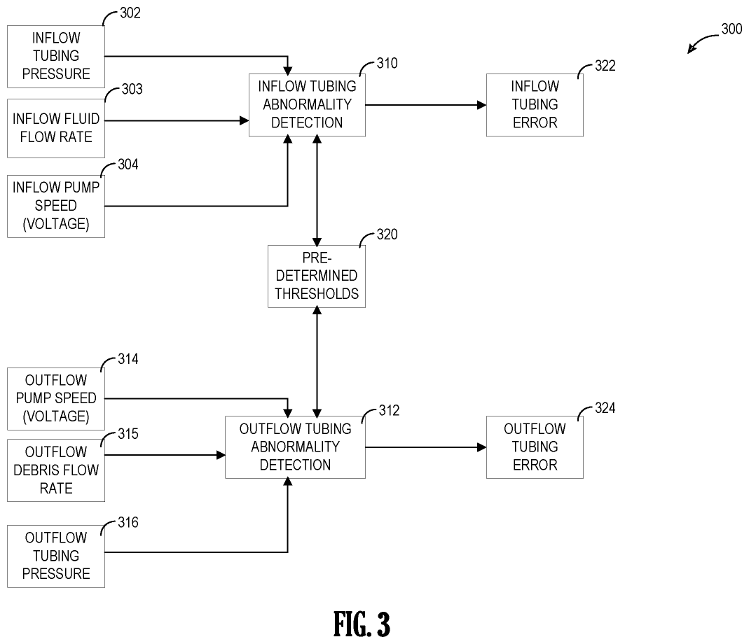

FIG. 3 is a schematic block diagram of an example of an abnormality detection system 300 for the system 200 (FIG. 2). The abnormality detection system 300 can include an inflow tubing abnormality detection module 310 and an outflow tubing abnormality detection module 312. The inflow tubing abnormality detection module 310 and the outflow tubing abnormality detection module 312 are shown as two separate components but they can be a single controller/processer (e.g., the control module 160 (FIG. 1) or the processing circuitry 234 (FIG. 2)). As shown in FIG. 3, the abnormality detection system 300 can include an inflow tubing pressure 302, an inflow fluid flow rate 303, and an inflow pump voltage 304. Each of the inflow tubing pressure 302, the inflow fluid flow rate 303, and the inflow pump voltage 304 can be detected by the inflow sensor 212 (FIG. 2) and sent to the abnormality detection system 300 as the inflow signal 216 (FIG. 2). Similarly, the abnormality detection system 300 can include an outflow tubing pressure 314, an outflow debris flow rate 315, and an outflow pump speed (outflow pump voltage 316), which can each be detected by the outflow sensor 218 (FIG. 2) and sent to the abnormality detection system 300 as the outflow signal 222 (FIG. 2).

The inflow tubing abnormality detection module 310 can compare either one, or both, of the inflow tubing pressure 302, the inflow fluid flow rate 303, and the inflow pump voltage 304 to respective pre-determined thresholds 320. Each of the inflow tubing pressure 302, inflow fluid flow rate 303, and the inflow pump voltage 304 can be indicative of fluid flowing through the inflow lumen 204 (FIG. 2). The outflow tubing abnormality detection module 312 can compare either one, or both, of the outflow tubing pressure 314, the outflow debris flow rate 315, and the outflow pump voltage 316 to the respective pre-determined thresholds 320. Each of the outflow tubing pressure 314, the outflow debris flow rate 315, and the outflow pump voltage 316 can be indicative of debris flowing within the outflow lumen 208 (FIG. 2).

As shown in FIG. 3, the inflow tubing abnormality detection module 310 can output an inflow tubing error 322 based upon detecting a possible abnormality when comparing the inflow tubing pressure 302, inflow fluid flow rate 303, or the inflow pump voltage 304 to their respective pre-determined thresholds 320. Similarly, the outflow tubing abnormality detection module 312 can output an outflow tubing error 324 based upon detecting a possible abnormality when comparing the outflow tubing pressure 314, the outflow debris flow rate 315, or the outflow pump voltage 316 to respective pre-determined thresholds 320.

In examples, the inflow tubing error 322 can be indicative of a kink, leak, or other issues (e.g., irrigation source liquid running out, or the like) in the inflow tubing 202 (FIG. 2) or the inflow lumen 204 (FIG. 2) and can alert the clinician of the error or adjust one or more components of the endoscopic system 100 to address the inflow tubing error 322. The outflow tubing error 324 can be indicative of a kink, leak, clog, blockage, or the like in the outflow tubing 206 (FIG. 2) or the outflow lumen 208 (FIG. 2) and can alert the clinician of the error or adjust one or more components of the endoscopic system 100 to address the outflow tubing error 324.

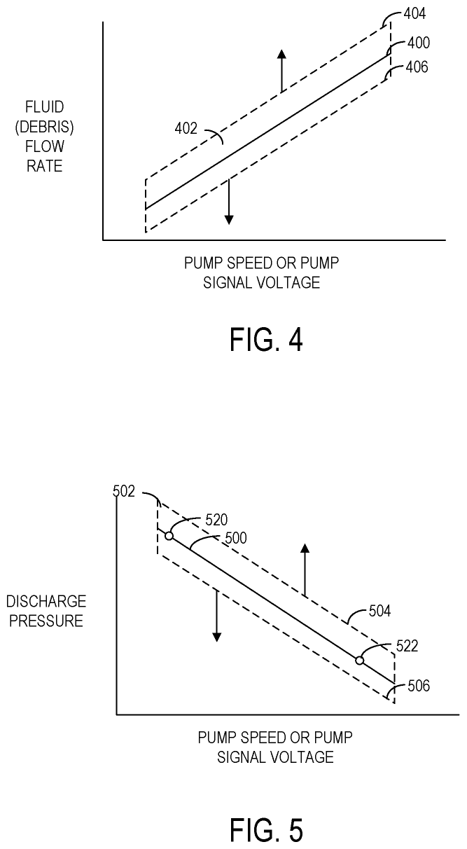

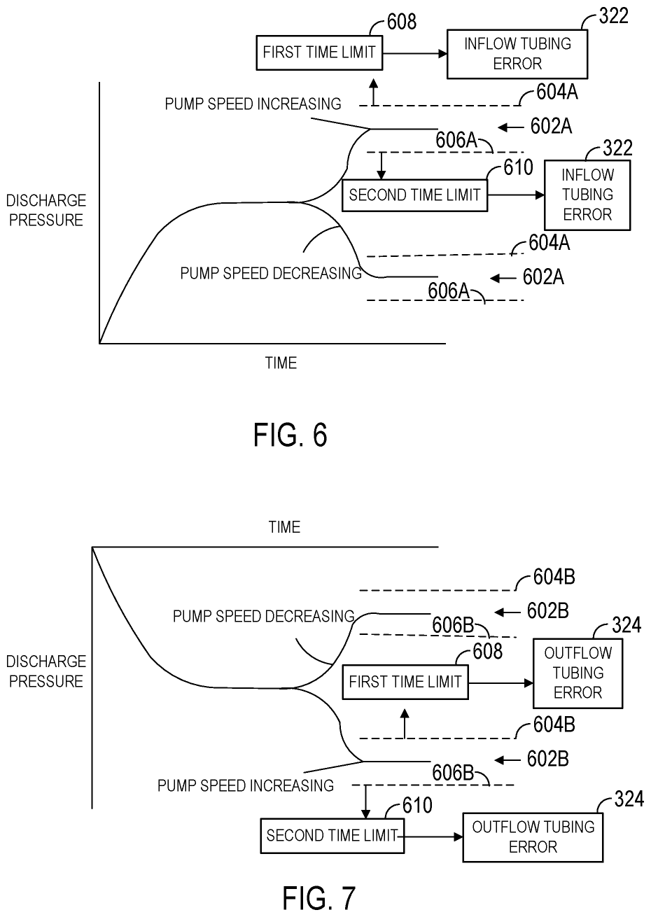

FIGS. 4-7 are graphical representations of the inflow pump and outflow pump, having a discharge pressure as a function of the pump speed or pump voltage. In the examples of FIGS. 4 and 5, the processing circuitry 234 (FIG. 2) is connected to the inflow sensor 212 (FIG. 2) and the outflow sensor 218 (FIG. 2). Each of the inflow sensor 212 and the outflow sensor 218 are configured to a fluid flow rate within the inflow lumen 204 (FIG. 2) and a debris flow rate in the outflow lumen 208 (FIG. 2), respectively. In examples, FIGS. 4 and 6 can representative graphical representations of the inflow tubing 202 (FIG. 2) and FIGS. 5 and 7 can be representative graphical representations for the outflow tubing 206 (FIG. 2).

As shown in FIG. 4, inflow reference line 400 is a linear relationship with a positive slope such that as the motor speed of the pump or the signal voltage (being sent to the pump) increases, the fluid flow rate within the lumen also increases. The system can include a buffer zone 402 on either side of the reference line 400, for example, above and below the inflow reference line 400. The buffer zone 402 can be defined by a maximum limit 404 and a minimum limit 406.

In examples, when the fluid flow rate at a known motor speed of the inflow pump or the signal voltage of an inflow pump exceeds the maximum limit 404 (e.g., is outside the buffer zone 402), the inflow tubing abnormality detection module 310 (FIG. 3) can generate an inflow tubing error 322 (FIG. 3) indicative of a leak in the tubing (e.g., the inflow tubing 202 (FIG. 2)). When the fluid flow rate at a known motor speed of the inflow pump or the signal voltage of an inflow pump is less than the minimum limit 406, the inflow tubing abnormality detection module 310 can generate the inflow tubing error 322 indicative of a kink in the tubing (e.g., the inflow tubing 202 (FIG. 2)).

Because the inflow reference line 400 is a fluid (or debris) flow rate as a function of the pump motor speed or the voltage sent to the pump motor the graphical representation shown in FIG. 4 can also work for the outflow lumen 208 because as the pump speed increases the debris flow rate within the outflow lumen 208 can also increase. Thus, when the fluid flow rate at a known motor speed of the outflow pump or the signal voltage of an outflow pump exceeds the maximum limit 404 (e.g., is outside the buffer zone 402), the outflow tubing abnormality detection module 312 (FIG. 3) or the processing circuitry 234 (FIG. 2) can generate an outflow tubing error 324 (FIG. 3) indicative of a leak in the tubing (e.g., the outflow tubing 206 (FIG. 2)). When the fluid flow rate at a known motor speed of the outflow pump or the signal voltage of an outflow pump is less than the minimum limit 406, the outflow tubing abnormality detection module 312 can generate the outflow tubing error 324 indicative of a kink in the tubing (e.g., the outflow tubing 206 (FIG. 2)).

As shown in FIG. 5, outflow reference line 500 is a linear relationship between discharge pressure as a function of pump speed or signal voltage sent to the pump. Here, for an inflow pump, the graph can have a similar relationship as FIG. 4, such that the slope of the reference line can include a positive slope. With the positive slope for the inflow pump, if a signal is detected above the maximum line of the buffer zone, the inflow tubing abnormality detection module 310 (FIG. 3) can generate an inflow tubing error 322 (FIG. 3) indicative of a kink in the inflow tubing 202 (FIG. 2). If a signal is detected below the minimum line of the buffer zone, the inflow tubing abnormality detection module 310 can generate the inflow tubing error 322 that can be indicative of a leak in the inflow tubing 202.

However, when the outflow tubing abnormality detection module 312 is comparing discharge pressure relative to the pump motor speed or pump voltage, the slope of the outflow reference line 500 can have a negative slope such that as the motor speed of the pump or the signal voltage (being sent to the pump) increases, the discharge pressure of the pump decreases, as shown in FIG. 5. The system can include a buffer zone 502 on either side of the reference line 500, for example, above and below the outflow reference line 500. The buffer zone 502 can be defined by a maximum limit 504 and a minimum limit 506.

In examples, when the discharge pressure at a known motor speed of the outflow pump or the signal voltage of an outflow pump exceeds the maximum limit 504 (e.g., is outside the buffer zone 502), the outflow tubing abnormality detection module 312 (FIG. 3) or the processing circuitry 234 (FIG. 2) can generate an outflow tubing error 324 (FIG. 3) indicative of a leak or leakage in the tubing (e.g., the outflow tubing 206 (FIG. 2)). When the discharge pressure at a known motor speed of the outflow pump or the signal voltage of an outflow pump is less than the minimum limit 506, the outflow tubing abnormality detection module 312 can generate the outflow tubing error 324 indicative of a tube kink or clogging or blockage within the outflow lumen (e.g., the outflow lumen 208) of the tubing (e.g., the outflow tubing 206 (FIG. 2)).

Calibration of the reference lines (e.g., the inflow reference line 400 or the outflow reference line 500) can be done completely off-site (e.g., at the factory or some third party before delivery of the endoscopic system 100 (FIG. 1) or the system 200 (FIG. 2), partially off-site, or on-site (e.g., after delivery of the endoscopic system 100 or the system 200 to the clinician. In examples, a first point 520 of the outflow debris flow rate reference line 500 can include a pre-set value and a second point 522 of the outflow debris flow rate reference line 500 can be set during set up of the system before or during the medical procedure.

As shown in FIGS. 6 and 7, as the pumps (e.g., the inflow pump 240 (FIG. 2) or the outflow pump 250 (FIG. 2)) the buffer zones 602A and 602B, respectively, can be applied to the reference lines as the speed of the pumps is increased or decreased. Moreover, a timing factor can be added to the buffer zones. In examples, if the pressure within the lumen exceeds the maximum limit 604A of the buffer zone 602A for a first time limit 608 or if the pressure within the lumen is below a minimum limit 606A of the buffer zone 602A for a second time limit 610, the inflow tubing abnormality detection module 310 (FIG. 3) or the processing circuitry 234 (FIG. 2) can generate an inflow tubing error 322 (FIG. 3). As such, different operating parameters (e.g., speeding up, slowing down, or maintaining at different pumping speeds) of the pump can result in the buffer zones 602A or 602B adjusting, the first-time limit 608 or the second time limit 610, or the like.

Thus, the system 200, and more specifically the processing circuitry 234 (FIG. 2), the inflow tubing abnormality detection module 310 (FIG. 3), or the outflow tubing abnormality detection module 312 (FIG. 3) can monitor one or more parameters of the inflow or the outflow (e.g., the inflow parameter 214 (FIG. 2) or the outflow parameter 220 (FIG. 2), respectively) to detect any abnormalities within the inflow lumen or the outflow lumen of the system.

As shown in FIG. 8, a first flow parameter threshold 804 can be a pre-determined flow parameter above or below a flow parameter reference line 802 at a third set buffer 822. A second flow parameter threshold 806 can be a pre-determined flow parameter above or below the flow parameter reference line 802 at a fourth buffer 824. In examples, the fourth set buffer 824 can have a linear relationship with the flow parameter reference line 802 such that the fourth set buffer 824 increases as the flow parameter reference line 802 increases such that as the flow parameter reference line 802 increases, the distance between the second flow parameter threshold 806 and the flow parameter reference line 802 increases.

The flow parameter reference line 802 can be a linear relationship made by at least two reference points (e.g., a first reference point 802A and a second reference point 802B). In examples, either of the first reference point 802A or the second reference point 802B can be pre-recorded by the manufacturer or a third party (e.g., before delivery of the system), such that no or little calibration is required. In another example, either of the first reference point 802A or the second reference point 802B can pre-recorded and the other of the first reference point 802A or the second reference point 802B can be found during calibration steps before or at the beginning of the medical procedure. Once, the first reference point 802A and the second reference point 802B are determined, the flow parameter reference line 802 can be extrapolated for an entire span of the range of speeds for the pump, and the first flow parameter threshold 804 and the second flow parameter threshold 806 can be set, respectively. Moreover, the system can adjust the first flow parameter threshold 804 or the second flow parameter threshold 806 during the medical procedure based on any flow parameter of the irrigation liquid or the debris (e.g., temperature, viscosity, flow rate, volume, or the like) to decrease the likelihood of a false alarm (inflow tubing error or outflow tubing error) as the flow parameters shift.

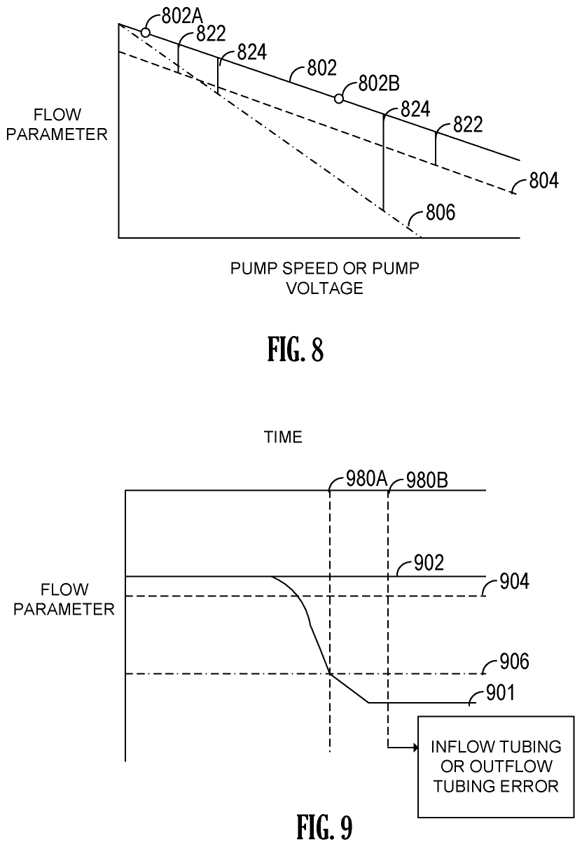

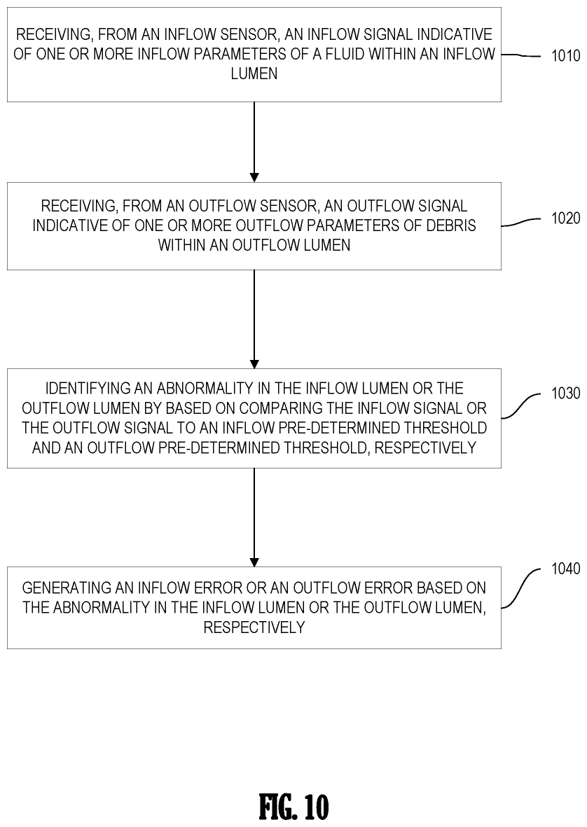

As shown in FIG. 9, a signal 901 (e.g., inflow signal 216 (FIG. 2) or the outflow signal 222 (FIG. 2)) can be tracked as a function of time. The signal 901 can be indicative of one or more flow parameters (e.g., inflow parameter 214 (FIG. 2) or the outflow parameter 220 (FIG. 2)). Once the signal 901 is beyond either of a first threshold value 904 or a second threshold value 906 for a set amount of time (difference between a first time 980A and a second time 980B), the system (e.g., the processing circuitry 234, the inflow tubing abnormality detection module 310, or the outflow tubing abnormality detection module 312) can generate an inflow tubing or outflow tubing error (e.g., the inflow tubing error 322 (FIG. 3) or the outflow tubing error 324 (FIG. 3)).

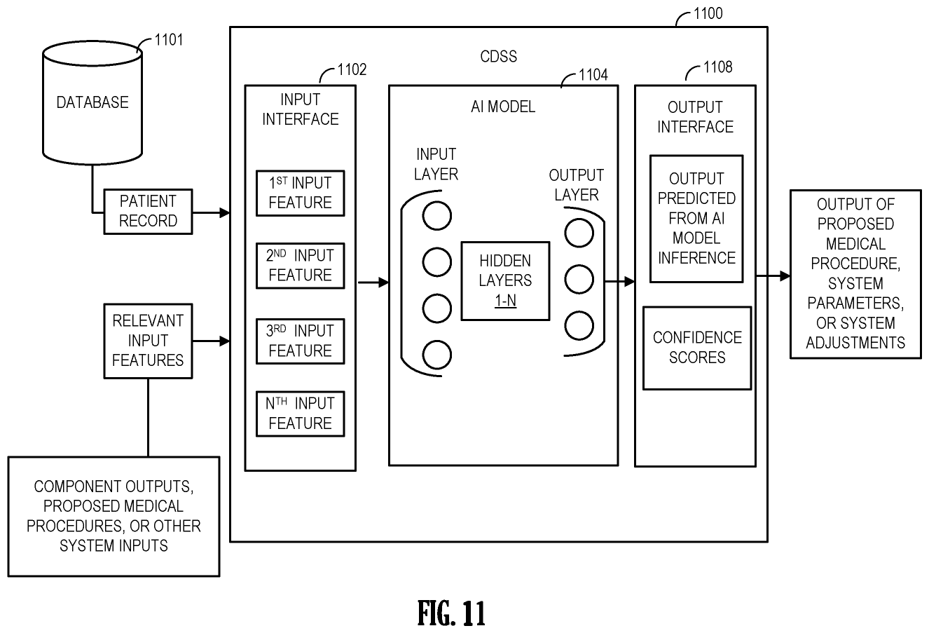

FIG. 10 is a schematic diagram of an example method 1000. The method 1000 can be a method for detecting abnormalities in a medical device during a medical procedure in a patient. The method can include any of operations 1010-1040.

At operation 1010, the method 1000 can include receiving, from an inflow sensor, an inflow signal indicative of one or more inflow parameters of a fluid within an inflow lumen. At operation 1020, the method 1000 can include receiving, from an outflow sensor, an outflow signal indicative of one or more outflow parameters of debris within an outflow lumen.

At operation 1030, the method 1000 can include identifying an abnormality in the inflow lumen or the outflow lumen by comparing the inflow signal or the outflow signal to an inflow pre-determined threshold and an outflow pre-determined threshold, respectively. At step 1040, the method 1000 can include generating an inflow error or an outflow error based on the abnormality in the inflow lumen or the outflow lumen, respectively.

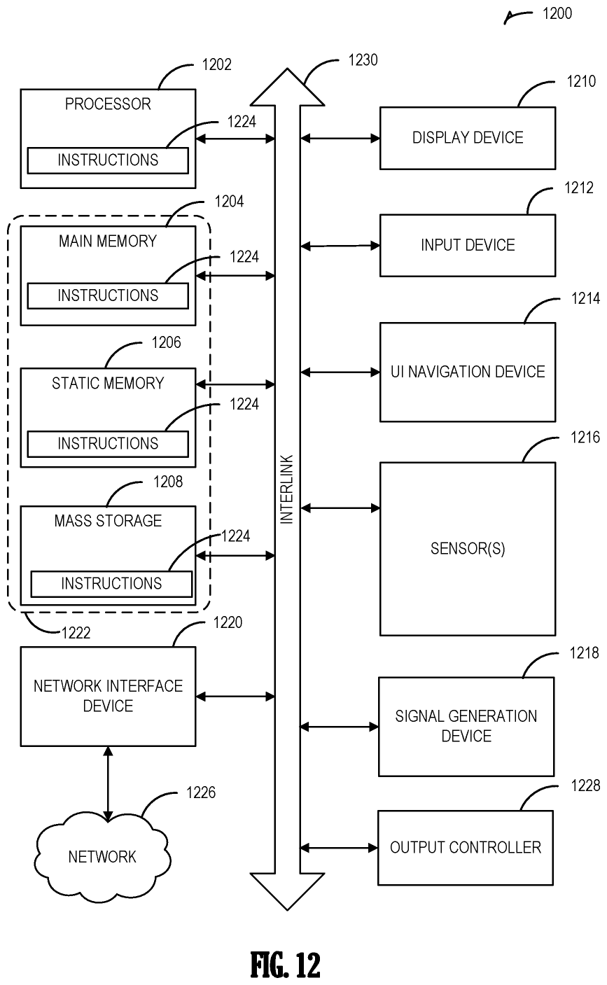

FIG. 11 shows a schematic diagram of an exemplary computer-based clinical decision support system (CDSS) 1100 (e.g., the CDSS 280 (FIG. 2)), which can be configured to control one or more aspects of the system (e.g., the endoscopic system 100 (FIG. 1), the system 200 (FIG. 2), or the like, based on input from any one of the components of the system (e.g., endoscope 110, the suction/irrigation control unit 140, the user interface 141, the control module 160, the pressure monitor 150, the inflow pump 158, or the outflow pump 159 (all in FIG. 1), any components of the system 200, or the like), signals from any of the inflow sensor 212, the outflow sensor 218, the inflow pump 240, the outflow pump 250, or the like). In examples, the CDSS 1100 can include an input interface 1102 through which medical information, such as, age, weight, sex, which are specific to a patient, or procedure specific information, such as, location within the patient of the planned medical procedure, planned path for the procedure, planned steps of the procedure, or the like, can be provided as input features to an artificial intelligence (AI) model 1104. A processor 1106 (e.g., the irrigation control unit 140 (FIG. 1), the processing circuitry 234 (FIG. 2), or the like) which can perform an inference operation in which the input from any one of the components of the endoscopic system or the fluid management system, signals transmitted based on either of the pumps (e.g., the inflow pump 240 or the outflow pump 250 (both in FIG. 2), medical information, procedure specific information, or the like, can be applied to the AI model to generate a suggested medical procedure, and a user interface (UI) (e.g., the display unit 260 (FIG. 2) through which suggested medical procedure is communicated to a user, e.g., a clinician.

In some embodiments, the input interface 1102 can be a direct data link between the CDSS 1100 and one or more medical devices (e.g., the endoscopic system 100 (FIG. 1), the system 200 (FIG. 2)), or the like, that can generate at least some of the input features. For example, the input interface 1102 can transmit input from any one of the components of the endoscopic system or the fluid management system, medical information, procedure specific information, or the like, directly to the CDSS 1100 during a therapeutic and/or diagnostic medical procedure. Additionally, or alternatively, the input interface 1102 can be a classical user interface that facilitates interaction between a user and the CDSS 1100. For example, the input interface 1102 can facilitate a user interface through which the user can manually enter medical information, procedure specific information, or the like. Additionally, or alternatively, the input interface 1102 can provide the CDSS 1100 with access to an electronic patient record from which one or more input features can be extracted. Such electronic patient records can be stored on a database 1101. In any of these cases, the input interface 1102 can be configured to collect one or more of the following input features in association with a specific patient on or before a time at which the CDSS 1100 is used to assess the safest and most efficient procedure to complete a planned medical procedure.

In examples, medical information, such as, age, weight, sex, which are specific to a patient, or procedure specific information, such as, location of anomaly can be provided to the CDSS 1100 via the input interface 1102.

In examples, medical procedure information, such as, the planned path for the procedure, planned steps of the procedure can also be provided to the CDSS 1100 via the input interface 1102.

In examples, any one of the components of the endoscopic system (e.g., endoscope 110, the suction/irrigation control unit 140, the user interface 141, the control module 160, the pressure monitor 150, the inflow pump 158, or the outflow pump 159 (all in FIG. 1), any components of the system 200, or the like), can provide input to the CDSS 1100 via the input interface 1102.

In examples, signals transmitted by the pumps or sensors (e.g., the inflow sensor 212, the outflow sensor 218, the inflow pump 240, the outflow pump 250, any other component of the system 200, or the like) can provide input to the CDSS 1100 via the input interface 1102.

Based on one or more of the above input features, the processor 1106 performs an inference operation using the AI model 1104 to generate the safest and most efficient medical procedure to perform the medical task. For example, input interface 1102 can deliver any of the medical information, medical procedure information, outputs from any one of the components of the endoscopic system, or signals transmitted based on engagement with either of the first engagement member or the second engagement member into an input layer of the AI model 1604 which propagates these input features through the AI model 1104 to an output layer. The AI model 1104 can provide a computer system the ability to perform tasks, without explicitly being programmed, by making inferences based on patterns found in the analysis of data. The AI model 1104 explores the study and construction of algorithms (e.g., machine-learning algorithms) that can learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled and allowing the algorithm to act on that information without guidance. Unsupervised ML is useful in exploratory analysis because it can automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised-ML algorithms are K-means clustering, principal component analysis, and autoencoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model can be trained continuously or periodically prior to performance of the inference operation by the processor 1106. Then, during the inference operation, the patient specific input features provided to the AI model can be propagated from an input layer, through one or more hidden layers, and ultimately to an output layer that corresponds to the suggested medical procedure. For example, if the age of the patient, size of the patient, or any other medical information of the patient, and the medical information, such as, the location of the patient indicate the sample can be difficult to obtain, the processor 1106 can suggest a smaller version of the endoscope, suggest a different path that can maximize imaging and sampling efforts, or suggest a maximum energy used for any cutting, ablation, or removal procedures.

During and/or subsequent to the inference operation, the output interface 1108 can transmit any of the safest and most efficient medical procedure can be communicated to the user via the user interface (UI) and/or automatically cause any component of the endoscopic system for performing a desired action. For example, if the imaging quality is poor, the processor 1106 can transmit a signal to visual port 115 to alter the brightness, color, saturation, or any other light parameter, of the light transmitted, send a controlling signal to the irrigation control unit 140 (FIG. 1) to change a fluid supplied to the pump(s)), send a signal to the pump(s) (e.g., the inflow pump 240 (FIG. 2)) to alter a velocity or volume of fluid supplied to the imaging site, send a signal to the pump (e.g., the outflow pump 250 (FIG. 2)) to increase or decrease an amount of suction provided to the imaging site. These are exemplary actions that can be taken by the CDSS 1100 to aid in the instruction and procedure of the medical procedure. However, the inventors of the present application have contemplated how the CDSS 1100 can help with any aspect of the medical procedure, such as planning preoperatively, performing intraoperatively, or analyzing the procedure postoperatively, or the like.

FIG. 12 illustrates a block diagram of an example machine 1200 upon which any one or more of the techniques (e.g., methodologies) discussed herein can perform. Examples, as described herein, can include, or can operate by, logic or a number of components, or mechanisms in the machine 1200. Circuitry (e.g., processing circuitry) is a collection of circuits implemented in tangible entities of the machine 1200 that include hardware (e.g., simple circuits, gates, logic, etc.). Circuitry membership can be flexible over time. Circuitries include members that may, alone or in combination, perform specified operations when operating. In an example, hardware of the circuitry can be immutably designed to carry out a specific operation (e.g., hardwired). In an example, the hardware of the circuitry can include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components can be used in more than one member of more than one circuitry. For example, under operation, execution units can be used in a first circuit of a first circuitry at one point in time and reused by a second circuit in the first circuitry, or by a third circuit in a second circuitry at a different time. Additional examples of these components with respect to the machine 1200 follow.

In alternative examples, the machine 1200 can operate as a standalone device or can be connected (e.g., networked) to other machines. In a networked deployment, the machine 1200 can operate in the capacity of a server machine, a client machine, or both in server-client network environments. In an example, the machine 1200 can act as a peer machine in peer-to-peer (P2P) (or other distributed) network environment. The machine 1200 can be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1200 can include a hardware processor 1202 (e.g., a central processing unit (CPU), a graphics processing unit (GPU), a hardware processor core, or any combination thereof), a main memory 1204, a static memory (e.g., memory or storage for firmware, microcode, a basic-input-output (BIOS), unified extensible firmware interface (UEFI), etc.) 1206, and mass storage 1208 (e.g., hard drives, tape drives, flash storage, or other block devices) some or all of which can communicate with each other via an interlink (e.g., bus) 1230. The machine 1200 can further include a display unit 1210, an alphanumeric input device 1212 (e.g., a keyboard), and a user interface (UI) navigation device 1214 (e.g., a mouse). In an example, the display unit 1210, input device 1212 and UI navigation device 1214 can be a touch screen display. The machine 1200 can additionally include a storage device (e.g., drive unit) 1208, a signal generation device 1218 (e.g., a speaker), a network interface device 1220, and one or more sensors 1216, such as a global positioning system (GPS) sensor, compass, accelerometer, or other sensor. The machine 1200 can include an output controller 1228, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1202, the main memory 1204, the static memory 1206, or the mass storage 1208 can be, or include, a machine readable medium 1222 on which is stored one or more sets of data structures or instructions 1224 (e.g., software) embodying or utilized by any one or more of the techniques or functions described herein. The instructions 1224 can also reside, completely or at least partially, within any of registers of the processor 1202, the main memory 1204, the static memory 1206, or the mass storage 1208 during execution thereof by the machine 1200. In an example, one or any combination of the hardware processor 1202, the main memory 1204, the static memory 1206, or the mass storage 1208 can constitute the machine readable media 1222. While the machine readable medium 1222 is illustrated as a single medium, the term “machine readable medium” can include a single medium or multiple media (e.g., a centralized or distributed database, and/or associated caches and servers) configured to store the one or more instructions 1224.

The term “machine readable medium” can include any medium that is capable of storing, encoding, or carrying instructions for execution by the machine 1200 and that cause the machine 1200 to perform any one or more of the techniques of the present disclosure, or that is capable of storing, encoding or carrying data structures used by or associated with such instructions. Non-limiting machine readable medium examples can include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media can include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 1222 can be representative of the instructions 1224, such as instructions 1224 themselves or a format from which the instructions 1224 can be derived. This format from which the instructions 1224 can be derived can include source code, encoded instructions (e.g., in compressed or encrypted form), packaged instructions (e.g., split into multiple packages), or the like. The information representative of the instructions 1224 in the machine readable medium 1222 can be processed by processing circuitry into the instructions to implement any of the operations discussed herein. For example, deriving the instructions 1224 from the information (e.g., processing by the processing circuitry) can include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 1224.

In an example, the derivation of the instructions 1224 can include assembly, compilation, or interpretation of the information (e.g., by the processing circuitry) to create the instructions 1224 from some intermediate or preprocessed format provided by the machine readable medium 1222. The information, when provided in multiple parts, can be combined, unpacked, and modified to create the instructions 1224. For example, the information can be in multiple compressed source code packages (or object code, or binary executable code, etc.) on one or several remote servers. The source code packages can be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 1224 can be further transmitted or received over a communications network 1226 using a transmission medium via the network interface device 1220 utilizing any one of a number of transfer protocols (e.g., frame relay, internet protocol (IP), transmission control protocol (TCP), user datagram protocol (UDP), hypertext transfer protocol (HTTP), etc.). Example communication networks can include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 502.11 family of standards known as Wi-Fi®, IEEE 502.15.4 family of standards, peer-to-peer (P2P) networks, among others. In an example, the network interface device 1220 can include one or more physical jacks (e.g., Ethernet, coaxial, or phone jacks) or one or more antennas to connect to the communications network 1226. In an example, the network interface device 1220 can include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1200, and includes digital or analog communications signals or other intangible medium to facilitate communication of such software. A transmission medium is a machine-readable medium.

The following, non-limiting examples, detail certain aspects of the present subject matter to solve the challenges and provide the benefits discussed herein, among others.

Example 1 is a system for detecting abnormalities in a medical device during a medical procedure in a patient, the system comprising: an inflow tubing defining an inflow lumen, the inflow lumen configured to provide a fluid from a fluid source to a site of the medical procedure; an inflow sensor in communication with the inflow lumen to sense an inflow parameter of the fluid within the inflow lumen, the inflow sensor configured to generate an inflow signal indicative of the inflow parameter of the fluid; an outflow tubing defining an outflow lumen, the outflow lumen configured to extract debris from the site of the medical procedure; an outflow sensor in communication with the outflow lumen to sense an outflow parameter of the debris within the outflow lumen, the outflow sensor configured to generate an outflow signal indicative of the outflow parameter of the debris; a memory including instructions; and processing circuitry that, when in operation, is configured by the instructions to: receive at least one of the inflow signal or the outflow signal; and identify an abnormality in either the inflow tubing or the outflow tubing based on comparing the inflow signal or the outflow signal, respectively, to pre-determined thresholds.

In Example 2, the subject matter of Example 1 optionally includes an inflow pump in communication with the inflow tubing to control a fluid flow rate of the fluid through the inflow lumen, wherein the inflow pump is controlled by adjusting an inflow pump voltage; and an outflow pump in communication with the outflow tubing to control a debris flow rate of the debris through the outflow lumen, wherein the outflow pump is controlled by adjusting an outflow pump voltage.

In Example 3, the subject matter of Example 2 optionally includes wherein the inflow signal and the outflow signal are indicative of an inflow fluid flow rate and an outflow debris flow rate, respectively.

In Example 4, the subject matter of Example 3 optionally includes wherein in response to detecting that the inflow fluid flow rate exceeds a first inflow fluid flow rate threshold, the processing circuitry is configured to: generate a first irrigation error, the first irrigation error indicative of a leak in the inflow tubing.

In Example 5, the subject matter of Example 4 optionally includes wherein in response to detecting the inflow fluid flow rate below a second inflow fluid flow rate threshold, the processing circuitry is configured to: generate a second irrigation error, the second irrigation error indicative of a kink in the inflow tubing.

In Example 6, the subject matter of Example 5 optionally includes wherein the first inflow fluid flow rate threshold is a first pre-determined flow rate above an inflow reference line at a first set buffer and the second inflow fluid flow rate threshold is a second pre-determined flow rate at a second set buffer below the inflow reference line, and wherein the inflow reference line is a predicted fluid flow rate during normal operating conditions.

In Example 7, the subject matter of any one or more of Examples 3-6 optionally include wherein in response to detecting the outflow debris flow rate exceeds a first outflow debris flow rate threshold, the processing circuitry is configured to: generate a first suction error, the first suction error indicative of a leak in the outflow tubing.

In Example 8, the subject matter of Example 7 optionally includes wherein in response to detecting the outflow debris flow rate below a second outflow debris flow rate threshold, the processing circuitry is configured to: generate a second suction error, the second suction error indicative of a clog or a kink in the inflow tubing.

In Example 9, the subject matter of Example 8 optionally includes wherein the first outflow debris flow rate threshold is a pre-determined debris flow rate above an outflow debris flow rate reference line at a third set buffer and the second outflow debris flow rate threshold is a pre-determined debris flow rate below the outflow debris flow rate reference line at a fourth set buffer, the fourth set buffer having a linear relationship with the outflow debris flow rate reference line such that a distance between the second outflow debris flow rate threshold and the outflow debris flow rate reference line increases as the outflow debris flow rate reference line increases.

In Example 10, the subject matter of Example 9 optionally includes wherein a first point of the outflow debris flow rate reference line includes a pre-set value, and wherein a second point of the outflow debris flow rate reference line is set during set up of the system before or during the medical procedure.

In Example 11, the subject matter of any one or more of Examples 2-10 optionally include wherein the inflow signal and the outflow signal are indicative of an inflow pump discharge pressure and an outflow pump discharge pressure, respectively.

In Example 12, the subject matter of Example 11 optionally includes wherein in response to detecting that the inflow pump discharge pressure exceeds a first inflow pump discharge pressure threshold, the processing circuitry is configured to: generate a first irrigation error, the first irrigation error indicative of a kink in the inflow tubing.

In Example 13, the subject matter of Example 12 optionally includes wherein in response to detecting the inflow pump discharge pressure below a second inflow pump discharge pressure threshold, the processing circuitry is configured to: generate a second irrigation error, the second irrigation error indicative of a leak in the inflow tubing.

In Example 14, the subject matter of Example 13 optionally includes wherein the first inflow pump discharge pressure threshold is a first pre-determined discharge pressure above an inflow reference line at a first set buffer, wherein the second inflow pump discharge pressure threshold is a second pre-determined discharge pressure at a second set buffer below the inflow reference line, and wherein the inflow reference line is a predicted pump discharge pressure during normal operating conditions.

In Example 15, the subject matter of any one or more of Examples 11-14 optionally include wherein in response to detecting the outflow pump discharge pressure exceeds a first outflow pump discharge pressure threshold, the processing circuitry is configured to: generate a first suction error, the first suction error indicative of a leak in the outflow tubing.

In Example 16, the subject matter of Example 15 optionally includes wherein in response to detecting the outflow pump discharge pressure below a second outflow debris pump discharge pressure threshold, the processing circuitry is configured to: generate a second suction error, the second suction error indicative of a kink or blockage in the outflow tubing.

In Example 17, the subject matter of Example 16 optionally includes wherein the first outflow pump discharge pressure threshold is a third set buffer below an outflow debris pump discharge pressure reference line and the second outflow debris pump discharge pressure threshold is a fourth buffer below the outflow debris pump discharge pressure reference line, and wherein the fourth set buffer increases as the outflow debris pump discharge pressure reference line increases such that a distance between the second outflow debris pump discharge pressure threshold and the outflow debris pump discharge pressure reference line increases as the outflow debris pump discharge pressure reference line increases.

In Example 18, the subject matter of Example 17 optionally includes wherein a first point of the outflow debris pump discharge pressure reference line includes pre-set values, and wherein a second point of the outflow debris pump discharge pressure reference line is set during set up of the system before or during the medical procedure.

In Example 19, the subject matter of any one or more of Examples 2-18 optionally include wherein the processing circuitry is configured to: transmit a controlling signal based on the abnormality measured in either the inflow tubing or the outflow tubing.

In Example 20, the subject matter of Example 19 optionally includes wherein the controlling signal alters a voltage supplied to the inflow pump to change an inflow pumping rate of the inflow pump.

In Example 21, the subject matter of any one or more of Examples 19-20 optionally include wherein the controlling signal alters a voltage supplied to the outflow pump to change an outflow pumping rate of the outflow pump.

In Example 22, the subject matter of any one or more of Examples 19-21 optionally include wherein the outflow tubing comprises: a debris trap configured to collect debris in the outflow tubing, and wherein the controlling signal opens the debris trap to clear the debris from the debris trap.

Example 23 is a method for detecting abnormalities in a medical device during a medical procedure in a patient, the method comprising: receiving, from an inflow sensor, an inflow signal indicative of one or more inflow parameters of a fluid within an inflow lumen; receiving, from an outflow sensor, an outflow signal indicative of one or more outflow parameters of debris within an outflow lumen; identifying an abnormality in the inflow lumen or the outflow lumen by based on comparing the inflow signal or the outflow signal to an inflow pre-determined threshold and an outflow pre-determined threshold, respectively; and generating an inflow error or an outflow error based on the abnormality in the inflow lumen or the outflow lumen, respectively.

In Example 24, the subject matter of Example 23 optionally includes transmitting, via a display unit, the inflow error or the outflow error to the clinician performing the medical procedure.

In Example 25, the subject matter of any one or more of Examples 23-24 optionally include generating a controlling signal to alter one or more components of the medical device to mitigate the kink, clog, or blockage causing the inflow error or the outflow error.

Example 26 is any element of any of Examples 1-25.

The above detailed description includes references to the accompanying drawings, which form a part of the detailed description. The drawings show, by way of illustration, specific examples that can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description, with each claim standing on its own as a separate embodiment. The scope of the examples should be determined with reference to the appended claims, along with the full scope of equivalents to which such claims are entitled.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device can also be sterilized using any other technique known in the art, including but limited to beta or gamma radiation, ethylene oxide, or steam.

1. A system for detecting abnormalities in a medical device during a medical procedure in a patient, the system comprising:
an inflow tubing defining an inflow lumen, the inflow lumen configured to provide a fluid from a fluid source to a site of the medical procedure;
an inflow sensor in communication with the inflow lumen to sense an inflow parameter of the fluid within the inflow lumen, the inflow sensor configured to generate an inflow signal indicative of the inflow parameter of the fluid;
an outflow tubing defining an outflow lumen, the outflow lumen configured to extract debris from the site of the medical procedure;
an outflow sensor in communication with the outflow lumen to sense an outflow parameter of the debris within the outflow lumen, the outflow sensor configured to generate an outflow signal indicative of the outflow parameter of the debris;
a memory including instructions; and
processing circuitry that, when in operation, is configured by the instructions to:
receive at least one of the inflow signal or the outflow signal; and
identify an abnormality in either the inflow tubing or the outflow tubing based on comparing the inflow signal or the outflow signal, respectively, to pre-determined thresholds.

2. The system of claim 1, comprising:
an inflow pump in communication with the inflow tubing to control a fluid flow rate of the fluid through the inflow lumen; and
an outflow pump in communication with the outflow tubing to control a debris flow rate of the debris through the outflow lumen.

3. The system of claim 2, wherein the inflow signal and the outflow signal are indicative of an inflow fluid flow rate and an outflow debris flow rate, respectively.

4. The system of claim 3, wherein in response to detecting that the inflow fluid flow rate exceeds a first inflow fluid flow rate threshold, the processing circuitry is configured to:
generate a first irrigation error, the first irrigation error indicative of a leak in the inflow tubing.

5. The system of claim 4, wherein in response to detecting the inflow fluid flow rate below a second inflow fluid flow rate threshold, the processing circuitry is configured to:
generate a second irrigation error, the second irrigation error indicative of a kink in the inflow tubing.

6. The system of claim 5, wherein the first inflow fluid flow rate threshold is a first pre-determined flow rate above an inflow reference line at a first set buffer and the second inflow fluid flow rate threshold is a second pre-determined flow rate at a second set buffer below the inflow reference line, and wherein the inflow reference line is a predicted fluid flow rate during normal operating conditions.

7. The system of claim 3, wherein in response to detecting the outflow debris flow rate exceeds a first outflow debris flow rate threshold, the processing circuitry is configured to:
generate a first suction error, the first suction error indicative of a leak in the outflow tubing.

8. The system of claim 7, wherein in response to detecting the outflow debris flow rate below a second outflow debris flow rate threshold, the processing circuitry is configured to:
generate a second suction error, the second suction error indicative of a clog or a kink in the inflow tubing.

9. The system of claim 8, wherein the first outflow debris flow rate threshold is a pre-determined debris flow rate above an outflow debris flow rate reference line at a third set buffer and the second outflow debris flow rate threshold is a pre-determined debris flow rate below the outflow debris flow rate reference line at a fourth set buffer, the fourth set buffer having a linear relationship with the outflow debris flow rate reference line such that a distance between the second outflow debris flow rate threshold and the outflow debris flow rate reference line increases as the outflow debris flow rate reference line increases.

10. The system of claim 9, wherein a first point of the outflow debris flow rate reference line includes a pre-set value, and wherein a second point of the outflow debris flow rate reference line is set during set up of the system before or during the medical procedure.

11. The system of claim 2, wherein the inflow signal and the outflow signal are indicative of an inflow pump discharge pressure and an outflow pump discharge pressure, respectively.

12. The system of claim 11, wherein in response to detecting that the inflow pump discharge pressure exceeds a first inflow pump discharge pressure threshold, the processing circuitry is configured to:
generate a first irrigation error, the first irrigation error indicative of a kink in the inflow tubing.

13. The system of claim 12, wherein in response to detecting the inflow pump discharge pressure below a second inflow pump discharge pressure threshold, the processing circuitry is configured to:
generate a second irrigation error, the second irrigation error indicative of a leak in the inflow tubing.

14. The system of claim 13, wherein the first inflow pump discharge pressure threshold is a first pre-determined discharge pressure above an inflow reference line at a first set buffer, wherein the second inflow pump discharge pressure threshold is a second pre-determined discharge pressure at a second set buffer below the inflow reference line, and wherein the inflow reference line is a predicted pump discharge pressure during normal operating conditions.

15. The system of claim 11, wherein in response to detecting the outflow pump discharge pressure exceeds a first outflow pump discharge pressure threshold, the processing circuitry is configured to:
generate a first suction error, the first suction error indicative of a leak in the outflow tubing.

16. The system of claim 15, wherein in response to detecting the outflow pump discharge pressure below a second outflow debris pump discharge pressure threshold, the processing circuitry is configured to:
generate a second suction error, the second suction error indicative of a kink or blockage in the outflow tubing.

17. The system of claim 16, wherein the first outflow pump discharge pressure threshold is a third set buffer below an outflow debris pump discharge pressure reference line and the second outflow debris pump discharge pressure threshold is a fourth buffer below the outflow debris pump discharge pressure reference line, and wherein the fourth set buffer increases as the outflow debris pump discharge pressure reference line increases such that a distance between the second outflow debris pump discharge pressure threshold and the outflow debris pump discharge pressure reference line increases as the outflow debris pump discharge pressure reference line increases.

18. The system of claim 17, wherein a first point of the outflow debris pump discharge pressure reference line includes pre-set values, and wherein a second point of the outflow debris pump discharge pressure reference line is set during set up of the system before or during the medical procedure.

19. The system of claim 2, wherein the processing circuitry is configured to:
transmit a controlling signal based on the abnormality measured in either the inflow tubing or the outflow tubing.

20. The system of claim 19, wherein the controlling signal alters a voltage supplied to the inflow pump to change an inflow pumping rate of the inflow pump.

21. The system of claim 19, wherein the controlling signal alters a voltage supplied to the outflow pump to change an outflow pumping rate of the outflow pump.

22. The system of claim 19, wherein the outflow tubing comprises:
a debris trap configured to collect debris in the outflow tubing, and wherein the controlling signal opens the debris trap to clear the debris from the debris trap.

23. A method for detecting abnormalities in a medical device during a medical procedure in a patient, the method comprising:
receiving, from an inflow sensor, an inflow signal indicative of one or more inflow parameters of a fluid within an inflow lumen;
receiving, from an outflow sensor, an outflow signal indicative of one or more outflow parameters of debris within an outflow lumen;
identifying an abnormality in the inflow lumen or the outflow lumen by comparing the inflow signal or the outflow signal to an inflow pre-determined threshold and an outflow pre-determined threshold, respectively; and
generating an inflow error or an outflow error based on the abnormality in the inflow lumen or the outflow lumen, respectively.

24. The method of claim 23, comprising:
transmitting, via a display unit, the inflow error or the outflow error to a clinician performing the medical procedure.

25. The method of claim 23, comprising:
generating a controlling signal to alter one or more components of the medical device to mitigate the abnormality causing the inflow error or the outflow error.
